(19) United States
(12) Patent Application Publication
(10) Pub. No.: US 2024/0062892 A1
(43) Pub. Date: Feb. 22, 2024

(54) DEVICE FOR THE COMBINED DISPLAY OF DATA RELATING TO BLOOD TREATMENT MACHINES AND WATER PREPARATION SYSTEMS

(71) Applicant: FRESENIUS MEDICAL CARE DEUTSCHLAND GMBH, Bad Homburg (DE)

(72) Inventors: Jannik MÜLLER, Darmstadt (DE); Florian SCHWAIGER, Oberursel (DE); Norman FÖRSTER, Bad Vilbel (DE); Bernd LUNGHAMER, Frankfurt (DE)

(73) Assignee: FRESENIUS MEDICAL CARE DEUTSCHLAND GMBH, Bad Homburg (DE)

(21) Appl. No.: 18/259,417

(22) PCT Filed: Dec. 21, 2021
(86) PCT No.: PCT/EP2021/087078

§ 371 Date: Jun. 27, 2023

(30) Foreign Application Priority Data
Dec. 29, 2020 (DE) 10 2020 135 023.6

(51) Int. Cl. G16H 40/63; A61M 1/16

(52) U.S. Cl. CPC G16H 40/63; A61M 1/1656

(57) ABSTRACT

The present invention relates to a device for the combined display of data relating to at least one blood treatment machine and data relating to at least one water preparation system, wherein the device has a control unit that is programmed to consolidate different data formats of the data relating to the at least one blood treatment machine and the data relating to the at least one water preparation system and preferably to integrate the data and display them together, whereby a user receives indications with respect to an optimized operation of the at least one blood treatment machine and/or the at least one water preparation system. The present invention furthermore relates to a corresponding method.

The present invention relates to a device for the combined display of data relating to blood treatment machines and water preparation systems and to a method for the combined display of data relating to blood treatment machines and water preparation systems.

The control of the blood treatment machines and water preparation systems can be synchronized with one another and coordinated with one another, in particular in time, by the combined display of data relating to blood treatment machines and water preparation systems.

The blood treatment machines used and the required water technology are conventionally organized as separate machine parks and are also accommodated spatially separately in clinics and dialysis centers. This has the result that a staff member has to switch the water preparation systems on manually at the start of every working day and/or has to document the correct operation of the water preparation systems, for example the flow rates, the pressure, a chlorine content of the pure water produced, etc. Some water preparation systems can indeed be switched on and off automatically, but this only produces a slight increase in efficiency.

This procedure is very time consuming and frequently also imprecise since the water preparation systems are frequently switched on and operated manually at a fixed time and independently of an actual requirement of pure water. Energy and resources, for example in the form of water, concentrates, and salts, are hereby wasted. In addition, the service life of the water preparation systems is reduced due to the unnecessary operation.

Data relating to the water preparation systems and to the blood treatment machines are furthermore conventionally handled completely separately and in different data formats. This makes it difficult for a user to obtain an overview of, for example, the operating states of the water preparation systems and the blood treatment machines since the user would have to read different databases with different programs for this purpose that are optimized for the respective data format. There is usually no time for this in hectic daily routine of clinics so that inaccuracies in the coordination of the control of the water preparation systems and the blood treatment machines have to be accepted.

It is thus the underlying object of the present invention against this background, to alleviate or even to fully eliminate the problems of the prior art. It is in particular the underlying object of the present invention to enable a user to operate a system comprising at least one blood treatment machine and at least one water preparation system in as energy saving and/or resource saving a manner as possible.

This object is achieved by the subject matters of the independent claims. Advantageous further developments are the subject of the dependent claims.

A first aspect of the invention relates to a device for the combined display of data relating to at least one blood treatment machine and data relating to at least one water preparation system and/or concentrate mixing system, wherein the device has a control unit that is programmed to consolidate different data formats of the data relating to the at least one blood treatment machine and the data relating to the at least one water preparation system and/or concentrate mixing system and preferably to integrate the data and display them together, whereby a user receives indications with respect to an optimized operation of the at least one blood treatment machine and/or the at least one water preparation system and/or concentrate mixing system.

The present invention will be explained in the following for the example of a blood treatment machine and a water preparation system. Alternatively or additionally to the water preparation system, devices associated therewith such as a concentrate mixing system that serves the provision of dialysis liquid on the basis of the dialysis water produced can also be considered.

Worded more generally, in accordance with the invention, data relating to the consumption of a blood treatment machine (acts as a consumer), in particular a time period in which the blood treatment machine requires the production of dialysis water and/or dialysis liquid and/or an amount of the required liquid are put into context with the data of the water preparation system, concentrate mixing system, or similar (each act as suppliers).

In other words, data relating to the internal states present in the at least one blood treatment machine and in the at least one water preparation system are preferably displayed together to a user by means of a device in accordance with the invention, whereby the user is enabled to operate or to control the system comprising the at least one blood treatment machine and the at least one water preparation system correctly, in particular optimized with respect to energy consumption and/or resource consumption.

The operating states of the blood treatment machine and of the water preparation system can, for example, be coordinated in time with one another to prevent, for example, that the water preparation system is operated for the production of pure water even though there is no need for pure water on the part of the blood treatment machine, e.g. because no treatment is carried out.

A device in accordance with the invention is thus preferably configured to enable the user by a guided and dynamic man/machine interaction to optimize the operation of a system comprising at least one blood treatment machine and at least one water preparation system.

The data are preferably technical data that are provided with a time stamp and that reflect a state of the blood treatment machine or of the water preparation system, preferably an operating state, a consumption, a machine ID, a location, a routine of a treatment to be carried out, a presetting to start or to take up or to end an operating mode, a measurement value of a fluid (e.g. conductivity, flow, temperature), or any desired combination thereof.

The data relating to the blood treatment machine can reflect a machine ID, a location, a manufacturer of the machine, a serial number of the machine, a software version of the machine, an IP address of the machine, a module configuration of the machine, an operating mode (e.g. active or standby, treatment preparation [priming], function test, disinfection [by heat or chemically], flushing [i.e. cold water without chemical additives], reinfusion [treatment end], a treatment mode, a start or end time of a treatment, a start or an end time of a session, a service status, a list of the error messages produced during a session, or any desired combinations thereof.

The data relating to the water preparation system can relate either to the total water preparation system or a subcomponent thereof such as filter stage, a reverse osmosis system, a dichlorination unit, etc. and can, for example, reflect a machine ID, a location, a manufacturer of the machine, a serial number of the machine, a software version of the machine, an IP address of the machine, a module configuration of the machine, an operating mode (e.g. active or standby, ring disinfection, regeneration of the softening agent), a consumption (e.g. concentrate or salt), a conveying rate (e.g. a flow rate of produced pure water or a conveying rate of concentrate), a property of a fluid (e.g. a water temperature, a water hardness, or a chlorine content of the water produced), a pressure, for example in a line, or a weight of a tank (e.g. of a brine tank), or any desired combination thereof.

The data relating to the at least one blood treatment machine and the data relating to the at least one water preparation system are preferably transmitted in real time, so-to-say “live”, from the blood treatment machine and/or from the water preparation system or from a subcomponent thereof to a device in accordance with the invention. The data are at least transmitted to the device in accordance with the invention within a few seconds from the at least one blood treatment machine and from the at least one water preparation system. The data evaluated by the device in accordance with the invention are thus always up to date or at least as up to date as technically possible. The at least one blood treatment machine and the at least one water preparation system can be equipped for this purpose with at least one or more online measuring sensors.

In accordance with an advantageous embodiment, a device in accordance with the invention is furthermore configured to determine or predict a requirement of the at least one blood treatment machine for water produced by means of the at least one water preparation system on the basis of the data relating to the at least one blood treatment machine and to estimate on the basis of the data relating to the at least one water preparation system whether an amount of water produced by the water preparation system and/or a time period in which the water preparation system is operated for the production of pure water corresponds to the need or falls within a tolerance range encompassing it.

The operation of the water preparation system to produce pure water should, for example, ideally be started approximately 40 minutes before the start of a blood treatment, should continue during the blood treatment, and should be continued for approximately 10 minutes after the end of the treatment.

If the operating time period of the water preparation system for the production of pure water differs from the requirement of the blood treatment machine, for example because the water preparation system is switched on by routine at 7 a.m., but the first blood treatments are only carried out from 9 a.m. onward, this results in an unnecessary waste of energy and resources due to the operation of the water preparation system in the first 80 minutes of every working day.

A device in accordance with the invention can be configured or programmed to output a report to the user when the estimate shows that the water amount produced by the water preparation system and/or the time period in which the water preparation system is operated to produce pure water does/do not correspond to the requirement of the blood treatment machine or does/do not fall into a tolerance range encompassing it.

The tolerance range can be defined as desired, for example with respect to the time (a start of the water preparation system 40 minutes before the start of treatment is ideal, a start of the water preparation system 50 minutes before the start of treatment is tolerable) or with respect to the produced water amount (10 liters of pure water are required for the planned treatment; in the current setting the water preparation system produces 13 liters of pure water, the excess is tolerable).

The report can, for example, be displayed on the device in accordance with the invention itself, for example on a monitor in the manner of a dashboard, or can be output to an external end device, for example a smartphone or a tablet.

In accordance with an advantageous embodiment of the invention, the device is furthermore programmed to output a recommendation to the user to optimize the control of the at least one blood treatment machine and/or the at least one water preparation system, in particular with respect to the times of individual operating modes.

A dashboard is here to be understood as the term from information management and informatics by which a graphical user interface for visualizing data is designated. The dashboard is dynamic in the context of this invention due to the above-described update in a quasi-live manner.

This recommendation can, for example, also be displayed on the device in accordance with the invention itself, for example on a monitor in the manner of a dashboard, or can be output to an external end device, for example a smartphone or a tablet.

A device in accordance with the invention can furthermore be programmed to output at least one control instruction to the at least one blood treatment machine and/or to the at least one water preparation system.

The at least one control instruction is preferably issued or is made as part of a control or regulation based on the data relating to the at least one blood treatment machine and/or on the data relating to the at least one water preparation system. In other words, a feedback loop can be provided in which the operation of the blood treatment machine and/or of the water preparation system is regulated on the basis of the data determined. It is conceivable that the operation of the blood treatment machine and/or of the water preparation system is regulated automatically or fully automatically by means of the feedback loop. A device in accordance with the invention can have a control unit configured for this purpose.

The device can, for example, have an operating unit by means of which a user can input control instructions for the at least one blood treatment machine and/or the at least one water preparation system that are then output by the device in accordance with the invention to the at least one blood treatment machine and/or to the at least one water preparation system. The user can thus interact dynamically and directly with a system comprising the at least one blood treatment machine and/or the at least one water preparation system by means of the device in accordance with the invention or can control said system.

Alternatively or additionally, the device can furthermore be programmed to actuate an output with respect to the savings potential in costs and/or raw materials and/or energy associated with the output recommendation to optimize the control and/or with a corresponding control instruction to the at least one blood treatment machine and/or the at least one water preparation system. This provides the advantage that the user can directly see whether it is worthwhile to follow the recommendation of the device in accordance with the invention and, for example, to input a corresponding control instruction.

It would likewise be conceivable that a device in accordance with the invention enables a bidirectional communication between the at least one blood treatment machine and the at least one water preparation system.

The device in accordance with the invention could, for example, be programmed such that it accordingly sets the operation of the at least one water preparation system, preferably independently, for example, on the basis of the estimated requirement for pure water of the blood treatment machine or displays a corresponding suggestion of a control instruction that a user only has to release.

A device in accordance with the invention can furthermore be configured to electronically document, for example in an electronic log book, the data relating to the at least one blood treatment machine and/or the data relating to the at least one water preparation system or a subcomponent thereof.

Alternatively or additionally, a device in accordance with the invention can be configured to make a prediction on the basis of the data relating to the at least one blood treatment machine and/or the data relating to the at least one water preparation system or a subcomponent thereof as to when and which kind of service of the blood treatment machine and/or of the water preparation system is necessary. Deficiencies can thus be recognized at an early time and an efficient direct service can take place. The prediction can take place on the basis of empirical values of a day, a week, a month, several months, or also several years and can make use of data relating to machines and systems at different sites. The device thus, for example, prepares a specific recommendation for a specific optimization from a typical course over a week that typically always occurs on a specific weekday—for example because the treatments start later on Monday mornings or because fewer treatments frequently take place on Friday afternoons. In addition, environmental factors can play a role that, for instance, occur in the course of the years. If a typical temperature is higher because it is e.g. summer, a deviation in the average treatment time with a blood treatment machine can arise therefrom. Typical courses can also be produced over a month that can be taken into account on an improved recommendation by the device. For instance, typically occurring public holidays, service intervals of machines, supply fluctuations over a month.

Alternatively or additionally, a device in accordance with the invention can be configured such that it filters the data present from one, from a plurality, or from all the connected blood treatment machines such that the device only further processes those data for display that are not personal data or patient data.

A further aspect of the present invention relates to a method for the coordinated control of at least one blood treatment machine and of at least one water preparation system, comprising the steps
detecting data relating to the at least one blood treatment machine and data relating to the at least one water preparation system;
consolidating different data formats of the data relating to the at least one blood treatment machine and/or of the data relating to the at least one water preparation system and preferably integrating said data; and
displaying the data relating to the at least one blood treatment machine and the data relating to the at least one water preparation system on a common display device, preferably a device in accordance with the invention,
whereby a user receives indications with respect to an optimized operation of the at least one blood treatment machine and/or of the at least one water preparation system.

Alternatively or additionally to the water preparation system, a concentrate mixing system can also be considered from a technical data aspect.

The data relating to the blood treatment machine or to the water preparation system are preferably technical data that are provided with a time stamp and that reflect a state of the blood treatment machine or of the water preparation system, preferably an operating state, a consumption, a machine ID, a location, a routine of a treatment to be carried out, a presetting to take up or to end an operating mode, a measurement value of a fluid, or any desired combination thereof.

The method preferably furthermore comprises the steps:
determining or predicting on the basis of the data relating to the at least one blood treatment machine a requirement of the at least one blood treatment machine for water produced by means of the at least one water preparation system or a time period in which the blood treatment machine requires water produced by the water preparation system; and
estimating on the basis of the data relating to the at least one water preparation system whether a water amount produced by the water preparation system and/or a time period in which the water preparation system is operated to produce pure water corresponds to the requirement or falls within a tolerance range encompassing it.

The requirement or consumption of the at least one blood treatment machine can thus be determined or predicted with respect to a time period, for example an active time period of the water preparation system and/or of the concentration mixing system, and/or with respect to a volume or a quantity, preferably of the produced water and/or concentrate.

If the estimate shows that a difference is to be expected or is present between the operation of the at least one water preparation system and the requirement of the at least one blood treatment machine, an indication can be output to a user, for example by a message on a device in accordance with the invention, on a smartphone, or on a tablet.

A method in accordance with the invention can furthermore comprise the following step: outputting a recommendation to optimize the control of the at least one blood treatment machine and/or of the at least one water preparation system, in particular with respect to the time control of individual operating modes. The recommendation can, for example, be to delay the start time of an operation of the water preparation system so that the water preparation system is only switched on, for example, approximately 40 minutes before the start of the first blood treatment, whereby an unnecessary activity of the water preparation system is prevented.

As part of a device in accordance with the invention or of a method in accordance with the invention, such a recommendation can be made on the basis of empirical values of a day, a week, or also of a month, a year, or also a plurality of years.

Provision can furthermore be made as part of a method in accordance with the invention that at least one control instruction is output to the at least one blood treatment machine and/or to the at least one water preparation system, for example in that a user inputs a control instruction into a device in accordance with the invention or releases a recommendation.

A further optional step can furthermore be provided as part of a method in accordance with the invention in which the data present from a blood treatment machine are filtered such that only those data are processed in the method in accordance with the invention that are not personal data or patient data.

A further optional step can furthermore be provided as part of a method in accordance with the invention in which an improvement of the recommendation takes place on the basis of daily, weekly, monthly or yearly routines. The advantages and special features have already been described further above.

To make the results of a recommendation or control instruction clear to the user, a method in accordance with the invention can furthermore comprise the step: actuating an output with respect to the savings potential in costs and/or raw materials and/or energy associated with the output recommendation to optimize the control and/or with a corresponding control instruction to the at least one blood treatment machine and/or the at least one water preparation system.

The invention was described above by way of example with reference to at least one blood treatment machine and to at least one water preparation system. However, this only serves the description and not the restriction of the present invention. Any desired number of blood treatment machines can be provided and a plurality of water preparation systems can also be provided.

The at least one blood treatment machine is preferably a dialysis machine and the at least one water preparation system is preferably a system for producing dialysis water, for example by means of reverse osmosis. A dozen or more hemodialysis machines are frequently operated in a machine park in dialysis centers and hospitals.

Further advantages, features, and effects of the present invention result from the following description of preferred embodiments of the invention with reference to the Figures in which the same or similar components are marked by the same reference numerals. There are shown:

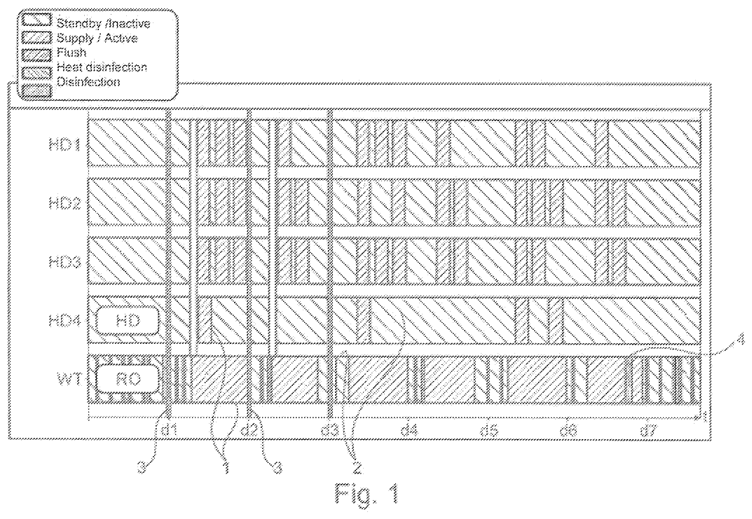

FIG. 1 a common display of the operating states of four blood treatment machines and of a water preparation system such as takes place, for example, by means of a device in accordance with the invention;

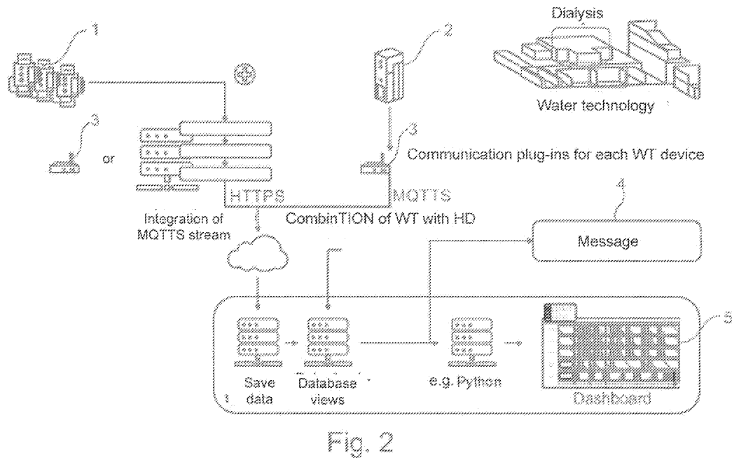

FIG. 2 a schematic overview of a method in accordance with the invention; and

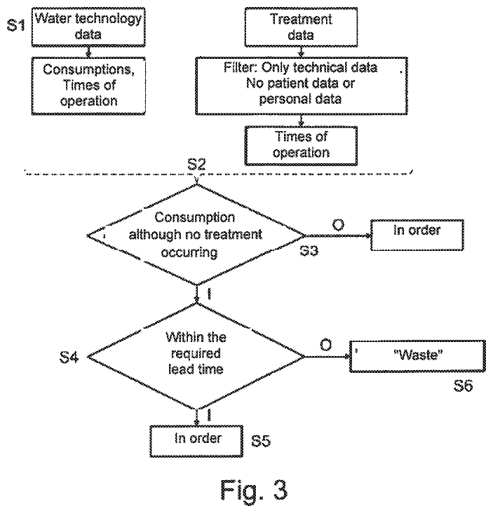

FIG. 3 a flow diagram of a method in accordance with the invention.

As shown in FIG. 1, the operating states of four blood treatment machines (the first four rows of the view in FIG. 1) and one water preparation system (the bottommost row in FIG. 1) are shown in a common view. In this example, the four blood treatment machines are hemodialysis machines (HD) and the water preparation system is a reverse osmosis system (RO). The X axis of the illustration represents the time development over a plurality of days. The operating states of the corresponding machine are shown in parallel with one another in every row.

The blocks marked by reference numeral 1 represent an operating state in which the corresponding machine is active, i.e. either produces pure water or carries out a blood treatment. The blocks marked by reference numeral 2 represent an operating state in which the corresponding machine is inactive or is in standby mode. A heat disinfection is carried out at the times marked by reference numeral 3. A disinfection of the water preparation system is carried out at the time marked by reference numeral 4.

The illustration shown in FIG. 1 makes it possible thus makes it possible for a user to be provided with an overview of the operating states of the system comprising the four blood treatment machines and the water preparation system at a glance.

FIG. 2 shows a schematic overview of a method in accordance with the invention. Data in this example relating to three blood treatment machines 1 and one water preparation system 2 are first determined.

The data of the three blood treatment machines 1 are either provided via a communication module 3 or a server in a first data format, in this example in the XML format, and are transferred to the cloud via Hypertext Transfer Protocol Secure (HTTPS).

The data of the water preparation system 2 are provided via a communication module 3 in a second data format and are transferred to the cloud via Message Queuing Telemetry Transport (MQTTS).

The first and second data formats differ. Which data format is used is arbitrary and is based e.g. on the conventions and regulatory requirements of a country in which the machines are located.

The data streams of the three blood treatment machines 1 and of the water preparation system 2 are associated with or linked to one another in the cloud, for example by means of a mapping process.

The data streams of the three blood treatment machines 1 (“HD” in FIG. 2) and of the water preparation system 2 (water technology “WT” in FIG. 2) are preferably converted into a common data format, for example in that views are generated in a common database. These views can then each be displayed on a dynamic dashboard. The database can, for example, be an SQL database and SQL views can then be prepared. Other database formats and database types are equally possible.

An output of an indication 4 to a user can take place on the basis of the combined data of the three blood treatment machines 1 and of the water preparation system 2, for example in that a user receives a message on an external end device such as a smartphone or a tablet.

Alternatively or additionally, the indication can also be such that information with respect to the operating state of the water preparation system, in particular an error message, is displayed, for example on a display device of at least one of the blood treatment machines. In this manner, a user of the blood treatment machine receives information with respect to the water preparation system at a glance without having to leave the site of the blood treatment machine.

FIG. 3 shows a flow diagram of a method in accordance with the invention. In Step S1, data relating to a water preparation system are collated in a second data format and data relating to a blood treatment device are collated in the XML format.

The data relating to a water preparation system indicate consumptions of the water preparation system (in particular a consumption with respect to energy, water, softener salt, dialyzate concentrate, or also a consumption in the sense of a wear of components, for example in the form of an operating time of the system, in particular an operating time of a reverse osmosis membrane or a prefilter) and times of the operation or the operating states.

The data relating to a blood treatment system preferably indicate times of the operation or of the operating states, for example for carrying out a treatment. Technical data are preferably exclusively logged, i.e. no sensitive data such as patient data and/or personal data are preferably collated or are only collated when the data owner—e.g. the patient—has previously explicitly consented.

The data relating to at least one water preparation system and the data relating to at least one blood treatment system are converted into a common data format and are linked to one another in Step S2.

A check is thereupon made in Step S3 whether the data relating to the at least one water preparation system indicate a consumption, for example because the water preparation system is active, even though the data relating to at least one blood treatment machine do not indicate any requirement, for example because the blood treatment machine is inactive.

A check is then made in Step S4 whether the operation of the water preparation system falls within a predetermined temporal tolerance range that, for example, starts approximately 40 minutes before the start of a blood treatment and ends approximately 10 minutes after the end of the blood treatment.

If the check in Step S4 shows that the operation of the water preparation system falls within a predetermined temporal tolerance range, a conclusion is drawn in Step S5 that the operation of the water preparation system is in order.

If the check in Step S4 shows that the operation of the water preparation system does not fall within a predetermined temporal tolerance range, for example because the water preparation system is also still active 30 minutes after the end of treatment, a conclusion is drawn in Step S6 that the operation of the water preparation system is not in order since resources are wasted unnecessarily.

Method Steps S2 to S6 are preferably displayed to a user on a device in accordance with the invention that has the form of a dynamic dashboard in this embodiment.

The display is preferably dynamic in the sense that the data used in Step S2 are always up to date and preferably show the state of the at least one water preparation system and/or of the at least one blood treatment system in real time, but at least with a delay of a few seconds, in particular between 1 and 10 seconds.

Without the present invention, it would not be possible for a user to obtain such a current overview of the operating steps of the at least one water preparation system and of the at least one blood treatment machine since they are usually accommodated spatially separately from one another. The effects of control instructions that a user inputs are also preferably displayed in real time in accordance with the invention or at least with a delay of only a few seconds. The display is thus also dynamic in the sense that the effects of the interaction of the user with the system are directly forwarded to the user.

1. A device for the combined display of data relating to at least one blood treatment machine and data relating to at least one water preparation system, wherein the device has a control unit that is programmed to consolidate different data formats of the data relating to the at least one blood treatment machine and the data relating to the at least one water preparation system and preferably to integrate the data and display them together, whereby a user receives indications with respect to an optimized operation of the at least one blood treatment machine and/or the at least one water preparation system.

2. A device in accordance with claim 1, wherein the data are preferably technical data that are provided with a time stamp and that reflect a state of the blood treatment machine or of the water preparation system, preferably an operating state, a consumption, a machine ID, a location, a routine of a treatment to be carried out, a presetting to take up or to end an operating mode, a measurement value of a fluid, or any desired combination thereof.

3. A device in accordance with claim 1, wherein the device in accordance with the invention is furthermore configured to determine or predict a requirement of the at least one blood treatment machine for water produced by means of the at least one water preparation system on the basis of the data relating to the at least one blood treatment machine and to estimate on the basis of the data relating to the at least one water preparation system and/or concentrate mixing system whether an amount of water produced by the water preparation system and/or a time period in which the water preparation system is operated for the production of pure water corresponds to the need or falls within a tolerance range encompassing it.

4. A device in accordance with claim 1, wherein the device is furthermore programmed to output a recommendation to the user to optimize the control of the at least one blood treatment machine and/or the at least one water preparation system, in particular with respect to the times of individual operating modes.

5. A device in accordance with claim 1, wherein the device is furthermore programmed to output at least one control instruction to the at least one blood treatment machine and/or to the at least one water preparation system, wherein the control instruction is preferably issued on the basis of a control or regulation based on the data relating to the at least one blood treatment machine and/or on the data relating to the at least one water preparation system.

6. A device in accordance with claim 4, wherein the device is furthermore programmed to actuate an output with respect to the savings potential in costs and/or raw materials and/or energy associated with the output recommendation to optimize the control and/or with a corresponding control instruction to the at least one blood treatment machine and/or the at least one water preparation system.

7. A method for the coordinated control of at least one blood treatment machine and of at least one water preparation system comprising the steps
collating data relating to the at least one blood treatment machine and data relating to the at least one water preparation system;
consolidating different data formats of the data relating to the at least one blood treatment machine and/or of the data relating to the at least one water preparation system and preferably integrating said data; and
displaying the data relating to the at least one blood treatment machine and the data relating to the at least one water preparation system on a common display device,
whereby a user receives indications with respect to an optimized operation of the at least one blood treatment machine and/or of the at least one water preparation system.

8. A method in accordance with claim 7, wherein the data are preferably technical data that are provided with a time stamp and that reflect a state of the blood treatment machine or of the water preparation system, preferably an operating state, a consumption, a machine ID, a location, a routine of a treatment to be carried out, a presetting to take up or to end an operating mode, a measurement value of a fluid, or any desired combination thereof.

9. A method in accordance with claim 7, further comprising the steps:
determining or predicting on the basis of the data relating to the at least one blood treatment machine a requirement of the at least one blood treatment machine for water produced by means of the at least one water preparation system, preferably with respect to an amount to be provided and/or a required active time period of the water preparation system; and
estimating on the basis of the data relating to the at least one water preparation system whether a water amount produced by the water preparation system and/or a time period in which the water preparation system is operated to produce pure water corresponds to the requirement or falls within a tolerance range encompassed by it.

10. A method in accordance with claim 7, further comprising the step: outputting a recommendation to optimize the control of the at least one blood treatment machine and/or of the at least one water preparation system, in particular with respect to the time control of individual operating modes.

11. A method in accordance with claim 7, further comprising the step: outputting at least one control instruction to the at least one blood treatment machine and/or to the at least one water preparation system, wherein the at least one control instruction is preferably issued on the basis of a control or regulation based on the data relating to the at least one blood treatment machine and/or on the data relating to the at least one water preparation system.

12. A method in accordance with claim 10, further comprising the step: actuating an output with respect to the savings potential in costs and/or raw materials and/or energy associated with the output recommendation to optimize the control and/or with a corresponding control instruction to the at least one blood treatment machine and/or the at least one water preparation system.
